(19) United States
(12) Patent Application Publication
(10) Pub. No.: US 2025/0022542 A1
(43) Pub. Date: Jan. 16, 2025

(54) SPATIAL MAPPING OF OMICS DATA

(71) Applicant: CANON MEDICAL SYSTEMS CORPORATION, Otawara-shi (JP)

(72) Inventors: Ian POOLE, Edinburgh (GB); Owen ANDERSON, Edinburgh (GB)

(73) Assignee: CANON MEDICAL SYSTEMS CORPORATION, Otawara-shi (JP)

(21) Appl. No.: 18/350,792

(22) Filed: Jul. 12, 2023

(51) Int. Cl. G16B 45/00; G06T 3/00; G16B 50/30

(52) U.S. Cl. CPC G16B 45/00; G06T 3/10; G16B 50/30

(57) ABSTRACT

A medical information processing apparatus comprising: a memory; and processing circuitry configured to: receive omics data, the omics data comprising a plurality of values, wherein each value of the plurality of values is associated with a corresponding biomolecule of a plurality of biomolecules; calculate a respective distance between each pair of biomolecules from the plurality of biomolecules; apply a manifold learning method to the distances to obtain a respective position in a two-dimensional space mapped to each biomolecule of the plurality of biomolecules; adjust the positions to achieve a more even distribution of the positions over the two-dimensional space; and store, in the memory, a two-dimensional image format of display positions for each biomolecule of the plurality of biomolecules based on the adjusted positions.

### FIELD

The present invention relates to the field of spatial mapping of multidimensional data, in particular when applied to omics data.

### BACKGROUND

Omics studies relate to the various branches of science aimed at characterizing biological molecules in order to understand the structure and function of organisms. For example, transcriptomics is the analysis of gene expression data to determine the number and variety of RNA transcripts found in a biological sample.

Gene expression data in a transcriptome may be considered to comprise a normalized count of mRNA molecules found in a tissue sample (for example, a colon polyp or a biopsied tumor). Using known relationships between DNA, RNA and proteins, a transcriptome may be considered to form a proxy for protein expression in the tissue.

A transcriptome can be obtained through RNA sequencing methods that includes microarrays, RNA-seq or probe based assays using a library of targeted primers, such as Temp'O Seq.

A typical gene expression dataset from a sample may contain around 10,000 to 60,000 measurements. If only the RNA that codes for proteins (mRNA) are considered, and if the low expressing genes are filtered out, the dataset can be reduced to around 10,000 measurements. When inspecting the dataset, there is no obvious ordering of the transcriptome results. By default, they are often listed alphabetically by gene name. Ordering based on chromosome position is also possible, but this has little functional significance.

FIG. 1 illustrates extracts from an example of an individual transcriptome which is stored in a spreadsheet. Each gene is given a respective index number. Each gene has an associated normalized gene expression value. In practice, a single spreadsheet may store transcriptomes for a number of patients or other subjects.

Transcriptomes, particularly of tumor biopsies, are widely studied and have been shown to have prognostic value. For example, transcriptomes of excised polyps may be analyzed to provide better stratification in colon cancer screening.

A transcriptomic dataset consisting of gene expression values for a number of samples is typically processed on a cohort basis by differential gene expression analysis. This is often followed by gene set enrichment analysis and visualized in Volcano plots or heat maps. A Volcano Plot comprises a plurality of points where each point is representative of respective gene, plotted as fold-change vs p-value, in respect of a whole cohort. It is not a patient specific plot.

These methods may often be useful for understanding perturbations in individual genes and gene sets. However, these methods typically do not provide an easily interpretable visual representation of the whole dataset for all samples.

The human visual cortex and modern neural network models, which both comprise convolutional network architectures, are good at spotting patterns in 2D images in order to classify them. Images can be meaningfully compared if they are defined or structured in some absolute way. There is therefore a need to present omics data so that it can be meaningfully read by humans and modern neural network models.

### BRIEF DESCRIPTION OF THE DRAWINGS

Embodiments are now described, by way of non-limiting example, and are illustrated in the following figures, in which:

FIG. 1 shows extracts of a transcriptome in a spreadsheet;

FIG. 2 is a schematic diagram of an apparatus according to an embodiment;

FIG. 3 is a flow chart illustrating in overview a method in accordance with an embodiment;

FIG. 4 is an exemplary image showing points output by a manifold learning method;

FIG. 5 is an exemplary image showing points after adjustment of position;

FIG. 6 is an example of a gene map;

FIG. 7 is an example of a displaying image for six subjects; and

FIG. 8 is an example of a displaying image with mouse-over functionality.

### DESCRIPTION

Certain embodiments provide a medical information processing apparatus comprising: a memory; and processing circuitry configured to: receive omics data, the omics data comprising a plurality of values, wherein each value of the plurality of values is associated with a corresponding biomolecule of a plurality of biomolecules; calculate a respective distance between each pair of biomolecules from the plurality of biomolecules; apply a manifold learning method to the distances to obtain a respective position in a two-dimensional space mapped to each biomolecule of the plurality of biomolecules; adjust the positions to achieve a more even distribution of the positions over the two-dimensional space; and store, in the memory, a two-dimensional image format of display positions for each biomolecule of the plurality of biomolecules based on the adjusted positions.

Certain embodiments provide a medical information processing method, comprising: receiving omics data, the omics data comprising a plurality of values, wherein each value of the plurality of values is associated with a corresponding biomolecule of a plurality of biomolecules; calculating a respective distance between each pair of biomolecules from the plurality of biomolecules; applying a manifold learning method to the distances to obtain a respective position in a two-dimensional space mapped to each biomolecule of the plurality of biomolecules; adjusting the positions to achieve a more even distribution of the positions over the two-dimensional space; and storing, in a memory, a two-dimensional image format of display positions for each biomolecule of the plurality of biomolecules based on the adjusted positions.

Certain embodiments provide a medical information processing apparatus comprising: a memory; and processing circuitry configured to: receive omics data, the omics data comprising a plurality of values, wherein each value of the plurality of values is associated with a corresponding biomolecule of a plurality of biomolecules; calculate a respective distance between each pair of biomolecules from the plurality of biomolecules; apply a manifold learning method to the distances to obtain a respective position in a two-dimensional space mapped to each biomolecule of the plurality of biomolecules; and store, in the memory, a two-dimensional image format of display positions for each biomolecule of the plurality of biomolecules based on the positions; wherein the respective distance between each pair of biomolecules from the plurality of biomolecules is calculated based on information from a biological database.

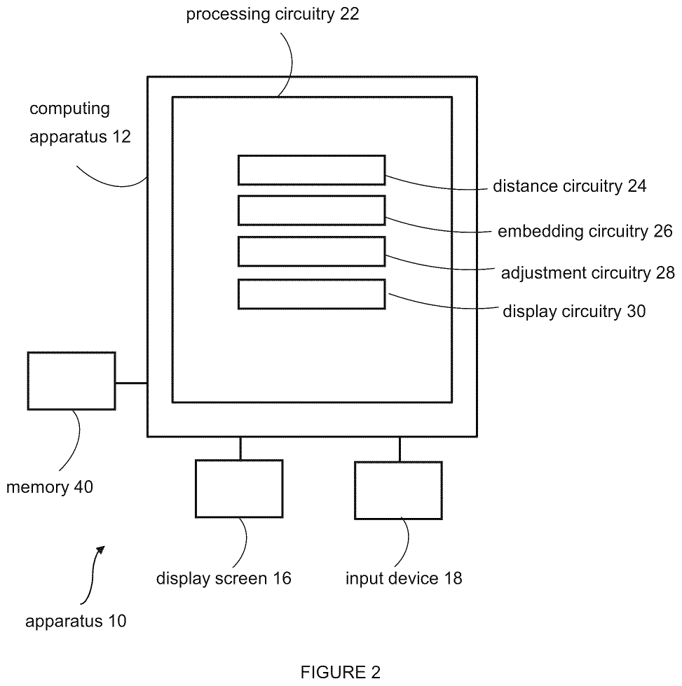

An apparatus 10 according to an embodiment is illustrated schematically in FIG. 2. The apparatus 10 may also be referred to as a medical information processing apparatus. The apparatus 10 is configured to process omics data. The apparatus 10 is further configured to display an image based on the omics data.

The omics data may be any type of omics data that comprises a plurality of values, wherein each value of the plurality of values is associated with a respective biomolecule of a plurality of biomolecules. The data can be, for example, transcriptome data, wherein the biomolecules are genes, and the associated values are expression levels of those genes. Alternatively, the omics data could be another gene related omics data, where the biomolecules are germline DNA, SNPs, or somatic mutations, and the associated values are, for example, a degree of mutational impact. Alternatively, the omics data may correspond to a biomolecule such as a protein or metabolite, with the associated values being protein or metabolite abundance respectively (i.e. the omics data is proteome or metabolome data).

In other embodiments, the apparatus 10 may be configured to process any appropriate data, which may comprise non-omics data, such as any unordered data. For instance, in some embodiments, the apparatus 10 may be configured to process any data comprising a plurality of values, wherein each value of the plurality of values is associated with a respective variable.

The apparatus 10 comprises a computing apparatus 12, which in this case is a personal computer (PC) or workstation. The computing apparatus 12 is connected to a display screen 16 or other display device, and an input device or devices 18, such as a computer keyboard and mouse. The computing apparatus 22 receives data from memory 40, which may also be referred to as a data store or storage. In alternative embodiments, computing apparatus 12 receives data from one or more further data stores (not shown) instead of or in addition to memory 40. For example, the computing apparatus 12 may receive data from one or more remote data stores (not shown), which may comprise cloud-based storage.

The memory 40 stores a two-dimensional image format of display positions for a plurality of biomolecules. The two-dimensional image format maps each of the plurality of biomolecules to pixel positions to be displayed as an image on display screen 16. The two-dimensional image format may be, for example, a matrix in which cells of the matrix are associated with respective biomolecules. In another example, the two-dimensional image format may be an image file format, for example a JPG or BMP file, in which pixel positions of an image are associated with respective biomolecules.

The memory 40 further stores omics data. In other embodiments, the omics data may be stored in another suitable memory, for example in another apparatus or in a cloud-based memory. The omics data can be stored in any file format suitable for storing text-based data such as TSV, CSV, XLS, XML.

Computing apparatus 12 comprises a processing circuitry 22 for processing data. The processing circuitry 22 comprises a central processing unit (CPU) and Graphical Processing Unit (GPU) and/or Tensor Processing Unit (TPU). The processing circuitry 22 provides a processing resource for automatically or semi-automatically processing omics data sets.

The processing circuitry 22 includes a distance circuitry 24 which is configured to, based on the plurality of biomolecules in the omics data, calculate a respective distance between each pair of biomolecules; an embedding circuitry 26 configured to map each of the plurality of biomolecules to a position based on the calculated distances; an image adjustment circuitry 28 configured to adjust the positions to obtain a two-dimensional image format; and a displaying circuitry 30 to display the omics data based on the two-dimensional image format.

In the present embodiment, the circuitries 24, 26, 28 and 30 are each implemented in the CPU and/or GPU and/or TPU by means of a computer program having computer-readable instructions that are executable to perform the method of the embodiment. In other embodiments, the circuitries may be implemented as one or more ASICs (application specific integrated circuits) or FPGAs (field programmable gate arrays).

The computing apparatus 12 also includes a hard drive and other components of a PC including RAM, ROM, a data bus, an operating system including various device drivers, and hardware devices including a graphics card. Such components are not shown in FIG. 2 for clarity.

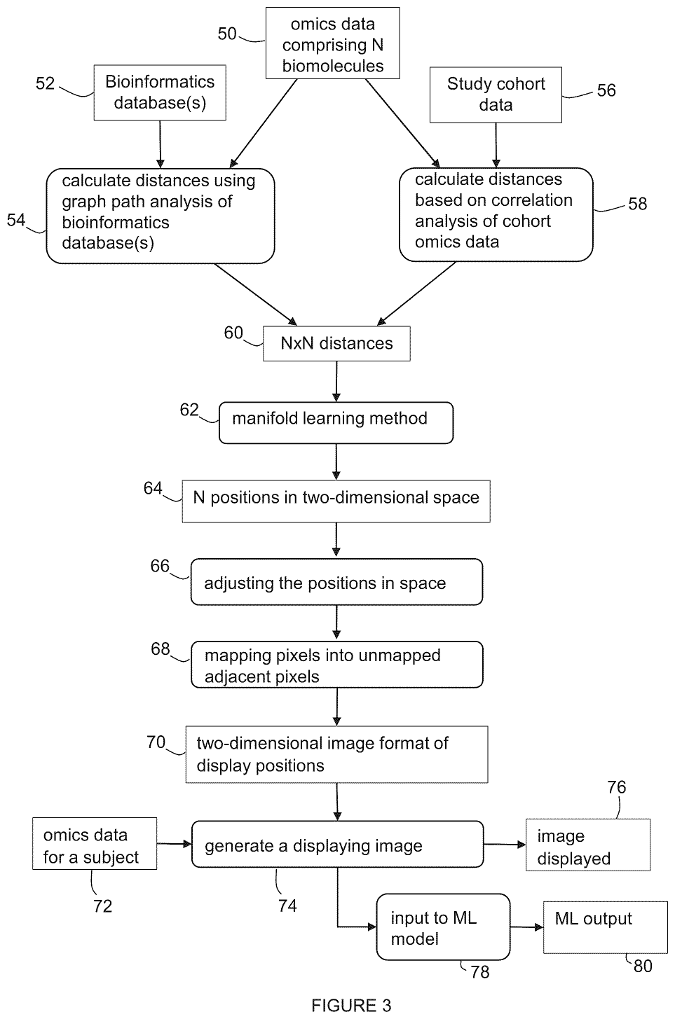

The processing circuitry 22 of FIG. 2 is configured to perform a method in accordance with FIG. 3. FIG. 3 is a flow chart illustrating in overview a method of generating a two-dimensional image format for a plurality of biomolecules and subsequently displaying an image based on the two-dimensional image format.

At stage 50, omics data is received by the distance circuitry 24 from the memory 40 or from any suitable data store. The omics data comprises a plurality of values, wherein each value of the plurality of values is associated with a respective biomolecule of a plurality of biomolecules. The plurality of biomolecules comprises N biomolecules. In the present embodiment, the biomolecules are genes that are associated with a respective gene expression value. In other embodiments, the omics data received by the distance circuitry 24 at stage 50 may comprise only a list of N biomolecules, for example a list of N genes, without also comprising values associated with the N biomolecules.

Stages 54 and 58 each provide a method of calculating distances between the N biomolecules. In some embodiments, stage 54 is performed and stage 58 is omitted. In other embodiments, stage 58 is performed and stage 54 is omitted. In further embodiments, stage 54 and stage 58, or outputs from stage 54 and stage 58, may be combined in any appropriate combination to obtain distances between the N biomolecules.

An input to stage 54 is data 52 from one or more bioinformatics databases, which may also be referred to as biological databases. A bioinformatics database or biological database may be a database that stores knowledge using a network, or knowledge graph, such as the STRING (Search Tool for the Retrieval of Interacting Genes/Proteins) or KEGG (Kyoto Encyclopedia of Genes and Genomes) database. In such a database, biomolecules are represented by nodes which are connected to each other by edges if the scientific literature indicates that they have an interaction. Edges may be weighted based on the strength of that interaction or based on the confidence in how likely that interaction is thought to be true.

At stage 54, distance circuitry 24 selects each pair of biomolecules from the plurality of biomolecules in turn and calculates a respective distance between each pair of biomolecules from the plurality of biomolecules using the data obtained from the bioinformatics database or databases. Distances are calculated for each possible pairing.

Using the STRING network as an example, which represents proteins by nodes and weights the edges by a confidence value, the distance between the two connected proteins may be calculated using 1—the confidence value. If two proteins are not directly connected, a distance may be found based on the minimum path distance between the two nodes.

A protein-protein network such as STRING may be used to determine distances between genes when those genes code for proteins. For example, when there are N genes, an N× N distance matrix may be calculated by, for each pair of genes defining a cell of the distance matrix, calculating a distance value between the two proteins that respective pair of genes code for, using information from the STRING network.

As an alternative to the STRING database, other databases may be used, for example databases that store connections between genes based on their co-expression, or databases that store connections between DNA, RNA or protein sequences based on sequence homology. Other databases may also be used which store connections between biomolecules based on their shared activity in biochemical pathways, or based on co-occurring mentions in academic papers.

The distance circuitry 24 outputs the distances 60 that were obtained at stage 54. The distances may be stored as a distance matrix of dimension N×N, where N is the number of biomolecules. Alternatively, the distances may be stored as a list or as any suitable data structure for storing numerical values.

An input to stage 58 is study cohort data 56 which comprises data, for example transcriptome data, for a plurality of individuals in a study cohort. For example, a cohort may comprise data relating to hundreds or thousands of patients or other subjects. Any suitable cohort data may be used.

At stage 58, distance circuitry 24 selects each pair of biomolecules from the plurality of biomolecules in turn and calculates a respective distance between each pair of biomolecules from the plurality of biomolecules using the data obtained from the study cohort. Distances are calculated for each possible pairing.

Distances between pairs of biomolecules are calculated by performing a correlation analysis. For example, for each pair of biomolecules, a respective correlation coefficient, such as the sample correlation coefficient, is calculated based on the values associated with that pair of biomolecules across the subjects in the cohort. Once the sample correlation coefficient is found, the distance is calculated by taking an inverse of the correlation coefficient. In this way, highly correlated biomolecules will have a correspondingly short distance between them. In other embodiments, any suitable method of obtaining a distance from cohort data may be used.

For example, for transcriptomics data comprising N genes, an N×N correlation matrix is generated comprising the correlation coefficients for each pair of genes, wherein high correlation values correspond to similar expression patterns between the respective pair genes. An N×N distance matrix is then generated by, for each cell of the distance matrix, taking the corresponding correlation coefficient from the correlation matrix and obtaining an inverse by calculating either:
1-correlation coefficient, or
1/correlation coefficient.

In other embodiments, any suitable measure of correlation may be used, and any suitable inverse may be used.

The distance circuitry 24 outputs the distances 60 that were obtained at stage 58. The distances may be stored as a distance matrix of dimension N×N, where N is the number of biomolecules. Alternatively, the distances may be stored as a list or as any suitable data structure for storing numerical values.

In some embodiments, the distances 60 are obtained by stage 54 alone. In other embodiments, the distances 60 are obtained by stage 58 alone. In further embodiments, the distances 60 are obtained by a combination of stage 54 and stage 58, or by combining outputs of stage 54 and stage 58.

At stage 62, the distance circuitry 24 passes the calculated distances to the embedding circuitry 26. The embedding circuitry 26 performs a mapping method, which in the present embodiment is a manifold learning method, to embed the plurality of biomolecules into two-dimensional space based on the calculated distances 60. In other embodiments, the mapping method used to embed the biomolecules in two-dimensional space may not comprise a manifold learning method. Any appropriate mapping method may be used.

The manifold learning method may be a method such as multidimensional scaling (MDS) algorithm which projects a set of elements from a higher-dimensional space to a lower-dimensional space (such as two-dimensional) while as far as possible preserving some defined distance between the elements. As a result, biomolecules that have a short calculated distance between them will also be positioned close to each other in the embedding.

The two-dimensional embedding assigns a position to each of the biomolecules of the plurality of biomolecules. The position is a position in two-dimensional space, for example expressed as an x, y co-ordinate value. Biomolecules that are correlated are expected to be assigned positions that are close together in the two-dimensional space.

In other embodiments, any suitable dimension reduction method may be used to obtain co-ordinate values in two-dimensional space for the biomolecules using the calculated distances, for example UMAP (Uniform Manifold Approximation and Projection) or t-SNE (Stochastic Neighbour Embedding).

In some embodiments in which the biomolecules are genes, a manifold learning method is applied to a N×N gene distance matrix to obtain a two-dimensional embedding which assigns a respective position in a two-dimensional space to each gene. The embedding preserves the gene-gene distances such that genes mapped close together are correlated. It is also envisaged that the manifold learning method may be applied to any other suitable data structure comprising the calculated distances.

The embedding circuitry 26 outputs an embedding comprising a set of two-dimensional co-ordinate values 64 comprising a respective two-dimensional co-ordinate value for each of the N biomolecules.

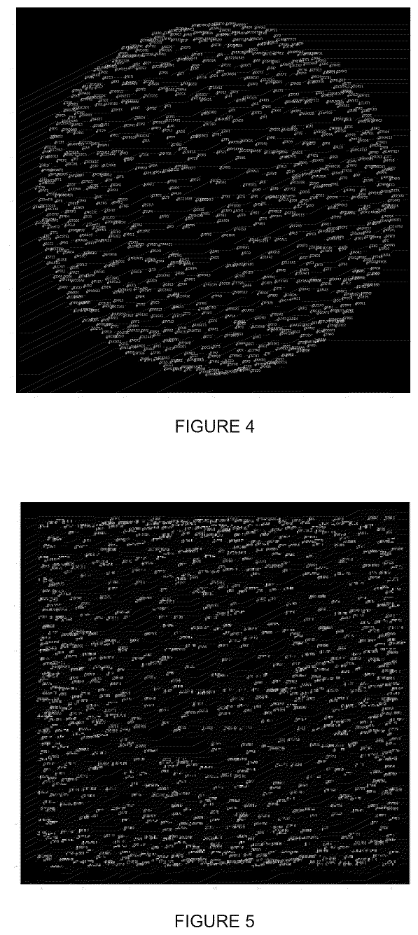

FIG. 4 illustrates an example of a set of two-dimensional co-ordinate values that are output from stage 64. In the example of FIG. 4, the biomolecules are genes, and the manifold learning method is MDS. The positions that are output by MDS are labelled with their associated genes.

This mapping between genes and positions may be used to show a transcriptome for a single patient or other subject, for example by coloring the points in dependence on whether the corresponding gene is under-expressed, over-expressed or little changed compared to cohort values. The coloring may be based on Z score, where a Z score indicates how many standard deviations a value is above or below the mean. In one example, points representing genes that are over-expressed are colored in red, points representing genes that are under-expressed are colored in blue, and points representing genes that are little changed are colored in grey.

In the embodiment illustrated in FIG. 4, the positions that are output by the MDS are restricted to an area defined by a circle. It is therefore the case that the positions that are output do not cover all of a square two-dimensional space. This can make it more difficult to visually inspect an image based on the embedding, since there will typically be many positions located at the perimeter of the circle, and no positions outside of the circle. The 2D placement of genes provides by MDS is unevenly distributed in the two-dimensional space.

At stage 66, adjustment circuitry 28 receives the embedding comprising the two-dimensional co-ordinate values 64 and adjusts at least some of the positions in the embedding to achieve a more even distribution of the positions over two-dimensional space. This may make it easier to visually inspect a resulting image.

In the present embodiment, the adjustment circuitry 28 receives x,y co-ordinate values and adjusts them so that they are more evenly distributed over two-dimensional space. In alternative embodiments, the adjustment circuitry 28 may map the x,y co-ordinate values to respective pixel positions and adjust the pixel positions so that they are more evenly distributed over an image.

The adjustment circuitry 28 adjusts the co-ordinate values or pixel positions, by, for example, applying an image distortion technique. The image distortion technique may be a spatial transformation, for example a square transformation. Once the positions are adjusted by the spatial transformation, they are more evenly spread across the whole of the two-dimensional space while, as much as possible, maintaining the relative distances between the positions output from MDS.

In other embodiments, the adjusting of the co-ordinate values or pixel positions may comprise perturbing the position corresponding to each biomolecule such that the adjusted positions lie equally spaced on a two-dimensional grid.

FIG. 5 shows an example of adjusted positions which are obtained by adjusting the positions shown in FIG. 4. In FIG. 4, the two-dimensional space is square and the unadjusted x, y co-ordinate values are distributed in a circular region of the two-dimensional space. The adjustment comprises a square transformation which distributes the positions over the whole of the square two-dimensional space. The adjustment may also comprise a non-linear radial scaling to account for clustering of positions at the periphery of the circle.

FIG. 5 shows the result of a square transformation that performs distortion of the two-dimensional co-ordinate values to fill a square. In the example of FIG. 5, the biomolecules are genes, and each adjusted position is labelled with its corresponding gene.

At stage 68, the adjustment circuitry 28 applies an image morphology method to the adjusted positions. If the adjusted positions are x,y co-ordinate values, the adjustment circuitry 28 first maps the co-ordinate values to respective pixel positions. If the adjusted positions are pixel positions, then the image morphology method is applied directly to the pixel positions.

Before the image morphology method is performed, each of the N biomolecules is mapped to a respective single pixel in two-dimensional image space. The number of pixels may be large compared to N, so there may be many pixels to which no biomolecule has yet been mapped. Such pixels may be described as unmapped pixels. If a gene is substantially unrelated to other genes, the pixel representing that gene may be surrounded by more unmapped pixels than a pixel representing a gene that has many related genes.

The adjustment circuitry 28 applies an image morphology method, such as dilation, to expand at least some of the mapped pixels into un-mapped adjacent pixels. A single biomolecule is initially mapped to a single pixel. After the image morphology method is applied, the biomolecules may be mapped to two, three, four, five or more pixels. The mapping is preserved such that each biomolecule is mapped to a group of adjacent pixels. This results in a map of biomolecules. When transcriptomics data is used, the resultant map is a gene map. The image morphology method may be such that, once the image morphology method has been performed, no unmapped pixels remain. In alternative embodiments, some unmapped pixels may be retained, for example to form borders between regions representing different biomolecules.

The adjustment circuitry 28 outputs a two-dimensional image format which comprises display positions for each biomolecule of the plurality of biomolecules. In the embodiment of FIG. 3, the display positions are pixel positions. The image format associates each biomolecule with a respective one or more pixels.

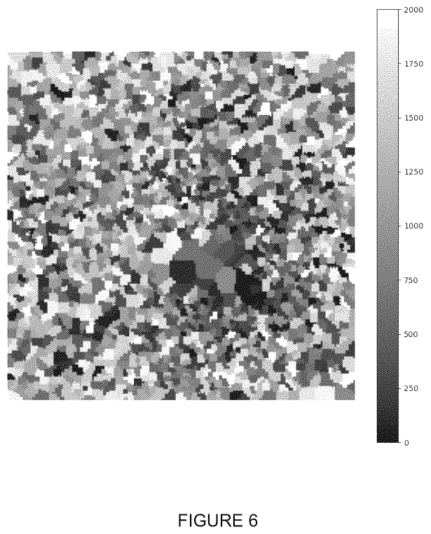

In some embodiments, the displaying circuitry 30 renders an index image, for example, a square gene map, using the display positions of the two-dimensional image format. In the index image, display positions (for example, pixels or groups of pixels) are colored in accordance with the gene (or other biomolecule) that they represent. FIG. 6 shows an example of a gene map which illustrates around 900 genes. A scale of the gene map represents gene index numbers by a color scale (represented in FIG. 6 by greyscale). Different colors represent different genes. Genes with sequential index numbers are not usually expected to be adjacent in the gene map, since index number is not indicative of correlation. Adjacent regions of the map often do not have similar colors. In other embodiments, no index image is rendered.

The two-dimensional image format is stored in memory 40. Alternatively, the two-dimensional image format may be stored in one or more remote data stores (not shown), which may comprise cloud-based storage.

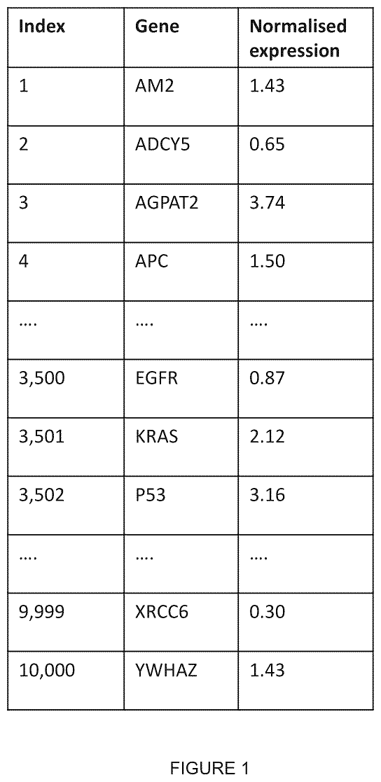

At stage 74, the display circuitry 30 receives the two-dimensional image format from memory 40 or any suitable data store. The display circuitry 74 also receives omics data 72 from memory 40 or any suitable data store. In the embodiment of FIG. 5, the omics data comprises data for a single patient or other subject, for example transcriptome data. In other embodiments, the omics data comprises data for multiple subjects. The omics data may be stored in a format as represented in FIG. 1.

The display circuitry 30 generates a displaying image 20 by, for each biomolecule of the plurality of biomolecules defined in the two-dimensional image format, using the associated value from the omics data to determine a color for the display position for that biomolecule. The colors may be determined using a color map which transforms the values associated with the biomolecules to a color scale. The displaying image 20 is rendered using the colors that are determined for all display positions in the displaying image 20. Values from the omics data are therefore mapped onto the displaying image based on the display positions defined in the two-dimensional image format.

In some embodiments, the values associated with the biomolecules are normalized values. For instance, if the omics data only comprises one sample, the values may be Z-score normalized based on all of the values comprised in that sample. If the omics data comprises more than one sample, the values associated with a specific biomolecule may be Z-score normalized based on values from all samples for that biomolecule. By displaying normalized values, it is easier to visually inspect the data to determine which biomolecules are over- or under-expressed, or activated, compared to an average. As an alternative, other normalization methods may be used such as mix-max scaling.

In the embodiment of FIG. 3, the omics data is transcriptome data and the color scale is representative of gene expression value. Gene expression values in the omics data, which are specific to a patient, are converted to Z-score values with respect to a cohort. The Z-score values are then used to determine color in the displaying image. For example, the color scale may color pixels that are representative of genes that are over-expressed in red, pixels that are representative of genes that are under-expressed in blue, and pixels representative of genes that are little changed in grey. In other embodiments, any suitable color scale may be used to represent any suitable values included in the omics data.

If the omics data comprises data from more than one subject, the displaying image 20 comprises a respective image for each patient, with the respective values for that patient mapped to the image. The same two-dimensional image format is used for each of the images. The two-dimensional image format therefore only has to be generated once for the omics data. Using the same two-dimensional image format for all of the images allows the data from each patient to be easily compared. For example, a position in two-dimensional that represents a given gene in a first image will also represent that same gene in a second, different image.

At stage 76, the display circuitry 30 displays the displaying image 20 on a display screen, for example the display screen 16. The displaying image 20 is displayed for human inspection.

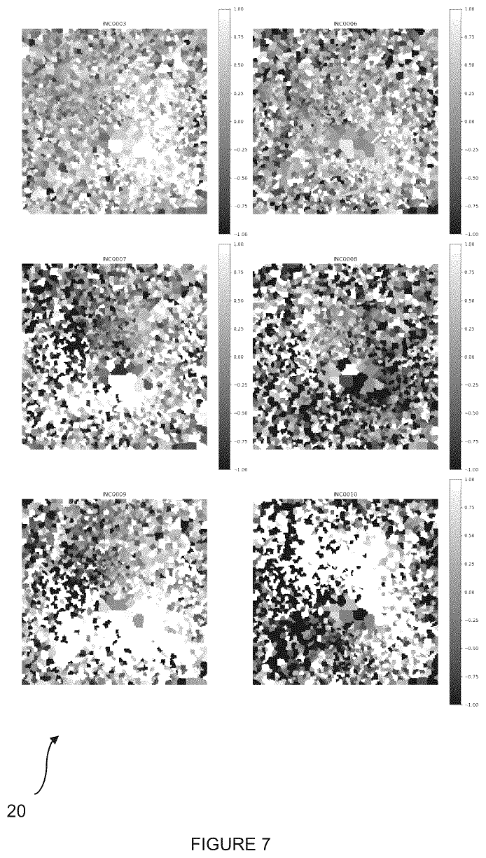

In one embodiment, as shown in FIG. 7, display circuitry 30 displays the images for each subject side by side, so that the data for each patient can be quickly compared. In

FIG. 7, six individual transcriptome images are rendered using the same two-dimensional image format. Colors (shown as greyscale in FIG. 7) are representative of over- or under-expression. Regions of over- and under-expression show coherence. The coherence may provide a further indication that related genes are mapped in proximity to each other. It may be seen that the images for the six patients are visibly different.

In another embodiment, the display circuitry 30 presents one image at a time, and provides the functionality to allow a user to switch between images in the same window, for example by using the input device 18 to provide an input that triggers display of a different image. For example, a different image may be displayed in response to a user input such as a click, a keystroke, or repositioning of a slider.

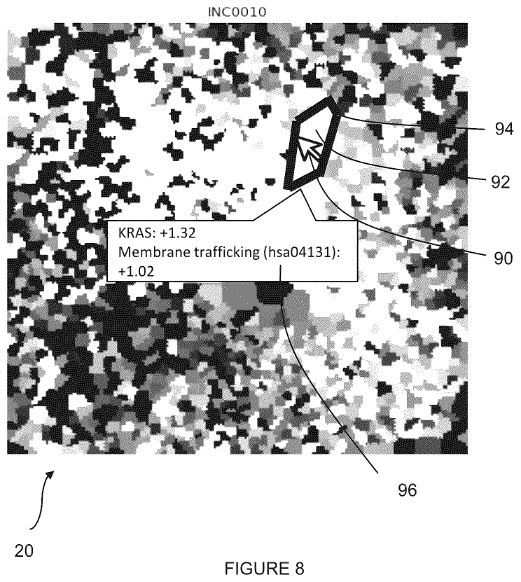

In some embodiments, as shown in FIG. 8, the display circuitry 30 adds mouse-over functionality to the displaying image 20. When a cursor 90, controlled by the input device 18, hovers over the display positions of a biomolecule, the display circuitry 30 receives an input that is indicative of the position of the cursor 90. The display circuitry 30 uses the two-dimensional image format to obtain the name of the biomolecule associated with the position of the cursor in the two-dimensional image format and/or to obtain functional information relating to that biomolecule. The functional information may be obtained from bioinformatics databases such as the Gene Ontology or KEGG ontology. The display circuitry 30 instructs display of the name and/or functional information on or beside the displaying image 20. In other embodiments, any suitable information may be displayed as a mouse-over display.

As an example, at stage 74, the display circuitry 30 receives from the memory 40 a two-dimensional image format which may also be described as gene map. The display circuitry 30 receives omics data 72 which comprises transcriptome data for multiple subjects. The display circuitry 30 generates a displaying image 20 for one of the subjects. Each of the mapped areas corresponding to genes in the displaying image 20 are colored based on the respective Z-score normalized gene expression values of those genes. The display circuitry 30 repeats this process for the other subjects and presents the images for each subject side by side for comparison. The display circuitry 30 adds mouse-over functionality to show individual gene names (e.g. KRAS, APS etc.) and functional interpretation of mapped genes using e.g. the Gene Ontology or KEGG ontology.

In some embodiments, different levels of hierarchy may be represented in the displaying image 20. For example, a functionality level in an ontology may be selected to identify a group of genes, and the display circuitry 20 may highlight a region that is representative of that group.

In the example shown in FIG. 8, the cursor 90 hovers over a region of pixels 92 that is representative of the KRAS gene. The display circuitry 30 highlights a boundary 94 of the region 92. The display circuitry 30 displays a text box 96 including text associated with the KRAS gene. In the example shown, the text is ‘KRAS: +1.32 Membrane trafficking (hsa04131): +1.02’. In other embodiments, any suitable text may be displayed that conveys any appropriate information. Any suitable display method may be used.

The displaying image 20 may provide a visually distinct representation of the omics data which may be considered to provide a gestalt of the data. When the omics data is transcriptomics data for a cohort, the displaying image 20 can be inspected to qualitatively see the difference between the subjects. Since genes that are plotted adjacently are highly correlated, regions of adjacent genes tend to show coherence. This means that there will be regions in the image that will be coherently under- or over-expressed across the cohort. It will therefore be possible, with practice, to become familiar with the co-expression profiles of certain genes in the dataset.

In some embodiments, a displaying image 20 may be used as a thumbnail image relating to a data set, for example a transcriptome data set. Use of a displaying image 20 as a thumbnail image may allow different data sets to be efficiently distinguished by eye.

It is a feature of the displaying image format that it exhibits coherence, in that neighboring pixels typically exhibit correlation. There is also typically coherence with respect to biological function, for example that neighboring pixels are likely to belong to the same Gene Ontology (GO) category and KEGG biochemical pathway. The image format is defined in an absolute way, so that transcriptome images from different sources can be meaningfully compared. The image format may be considered to be a constant map format that is reproducible.

In some embodiments, the display circuitry 20 performs stage 78 in addition to stage 76. In other embodiments, stage 78 is omitted. In further embodiments, stage 78 may be performed without stage 76 being performed.

At stage 78, the display circuitry 20 uses the displaying image 20 as input to a deep learning algorithm, such as a convolutional neural network (CNN) based machine learning model. The deep learning algorithm may be configured to perform any suitable operation on the displaying image 20. For example, the deep learning algorithm may be configured to classify the deep learning image. The deep learning algorithm processes the displaying image to obtain an output 80, for example a classification.

In the method of FIG. 3, unordered omics data is converted into an ordered displaying image 20. The conversion of unordered data into ordered data may allow the resulting ordered displaying image to be used as an input to a machine learning method that operates on images, such as a CNN. The machine learning method may be configured to perform any suitable task which may comprise any suitable processing of omics data.

In some embodiments, the displaying image 20 is augmented by parallel atlas images allowing the network to exploit non-stationary properties. Typically, a CNN may be considered to be blind to position within an image. The CNN may not make use of position within an image. However, in the case of omics data, it may be useful for position to be used, since biomolecules are represented at particular positions. Therefore, an atlas may be provided to the CNN along with the displaying image 20. The atlas may comprise or be derived from the two-dimensional image format. The atlas may provide a mapping from biomolecule names or other information to positions in the displaying image 20. The atlas may comprise a set of labelled x, y co-ordinates.

The method of FIG. 3 provides for omics data to be displayed as an image in a way that preserves the relationships between each of the biomolecules in a dataset. Biomolecules will be adjacent to each other in the displaying image 20 is they have a strong correlation based on the dataset or if they have some kind of functional relationship based on prior knowledge. The images offer a qualitative overview of the data, which can be used as an adjunct to other bioinformatics methods that are used to process data. The images can be used as an icon or preview for files or UI elements relating to the omics data. The images allow the data to be visually inspected by the user and once the user gains experience, the user might learn to recognize patterns for multiple images based on the same two-dimensional format.

Since the adjacency of neighboring biomolecules is meaningful, the images are suitable for CNN based ML models that can classify the images.

A two-dimensional image format (for example, a gene map) may be obtained that is data driven (by using cohort data) or knowledge driven (by using biological database data). In some circumstances, obtaining the two-dimensional image format from external knowledge may lead to more functionally interpretable image coherence, which may be cohort-independent.

In other embodiments, different apparatus may be used to perform different processes, or parts of processes, of those described below. For example, a first apparatus may be used to perform the distance calculation, a second apparatus may be used to perform the embedding, a third apparatus may be used to perform the image adjustment, and a fourth apparatus may be used to display the image. Any suitable combination of apparatuses may be used.

In some embodiments, a first apparatus is used to produce the two-dimensional image format. The two-dimensional image format is then used by one or more further apparatuses to generate displaying images from omics data. The two-dimensional image format may only need to be produced once for a given set of biomolecules, and then can be reused for displaying many data sets that comprise that set of biomolecules.

In some embodiments, one or more of the stages described above may be omitted, or multiple stages may be combined.

Certain embodiments provide a medical information processing apparatus comprising: a memory stores a two-dimensional image format in which display positions for the expression levels of a plurality of genes are defined based on interactions of the plurality of genes, and,
processing circuitry configured to:
receive a transcriptome data of a patient,
generate a displaying image by mapping the transcriptome data onto the two-dimensional image format based on the positions for the expression levels of a plurality of genes.

The display positions may be determined based on distances of between genes specified by the interactions of the plurality of genes.

Certain embodiments provide a method of presenting gene related omics data as a 2D image in which pixels have a fixed relationship to genes and adjacency has biological significance. The gene to pixel mapping may be driven by preserving distance defined as an inverse of gene interactions obtained from bioinformatics databases (e.g. STRING). Interaction distances may be obtained from correlation analysis of a cohort of the target omics data. A manifold learning algorithm (e.g. MDS) may be used to place the genes in 2D image space. The rectangular image map may be filled by image morphology methods which expand the placed genes into un-mapped adjacent pixels. The gene map may be used to render gene expression data (a transcriptome). The gene map may be used to render mutational data realised as mutational impact at each gene.

The gene expression values may be rendered according to Z-score with respect to a cohort of interest, using a suitable colour map. The rendered image may be animated so that mouse-over shows the boundaries of functional groups of genes.

Whilst particular circuitries have been described herein, in alternative embodiments functionality of one or more of these circuitries can be provided by a single processing resource or other component, or functionality provided by a single circuitry can be provided by two or more processing resources or other components in combination. Reference to a single circuitry encompasses multiple components providing the functionality of that circuitry, whether or not such components are remote from one another, and reference to multiple circuitries encompasses a single component providing the functionality of those circuitries.

Whilst certain embodiments have been described, these embodiments have been presented by way of example only, and are not intended to limit the scope of the invention. Indeed the novel methods and systems described herein may be embodied in a variety of other forms. Furthermore, various omissions, substitutions and changes in the form of the methods and systems described herein may be made without departing from the spirit of the invention. The accompanying claims and their equivalents are intended to cover such forms and modifications as would fall within the scope of the invention.

1. A medical information processing apparatus comprising:
a memory; and
processing circuitry configured to:
receive omics data, the omics data comprising a plurality of values, wherein each value of the plurality of values is associated with a corresponding biomolecule of a plurality of biomolecules;
calculate a respective distance between each pair of biomolecules from the plurality of biomolecules;
apply a manifold learning method to the distances to obtain a respective position in a two-dimensional space mapped to each biomolecule of the plurality of biomolecules;
adjust the positions to achieve a more even distribution of the positions over the two-dimensional space; and
store, in the memory, a two-dimensional image format of display positions for each biomolecule of the plurality of biomolecules based on the adjusted positions.

2. The medical information processing apparatus of claim 1, wherein the processing circuitry is further configured to:
after adjusting the positions and before storing the two-dimensional image format in the memory:
apply an image morphology method to adjust at least some mapped positions into un-mapped adjacent positions.

3. The medical information processing apparatus of claim 2, wherein the image morphology method comprises dilation.

4. The medical information processing apparatus of claim 1, wherein the positions are adjusted by applying a spatial transformation to the positions in two-dimensional space.

5. The medical information processing apparatus of claim 1, wherein the omics data comprises data from a cohort of subjects, and wherein the respective distance between each pair of biomolecules from the plurality of biomolecules is calculated based on a correlation between said pair of biomolecules across the cohort.

6. The medical information processing apparatus of claim 1, wherein the respective distance between each pair of biomolecules from the plurality of biomolecules is calculated based on information from a biological database.

7. The medical information process of claim 6, wherein the biological database is a knowledge graph.

8. The medical information processing apparatus of claim 7, wherein the knowledge graph defines biomolecules as nodes, wherein the nodes are connected by edges, and wherein the distance between each pair of biomolecules is calculated based on the number and/or weight of the edges between the respective biomolecules.

9. The medical information processing apparatus of claim 1, wherein the omics data is transcriptome data, the biomolecules are genes, and the associated values are expression levels of said genes.

10. The medical information processing apparatus of claim 1, wherein the omics data is one of proteome, metabolome, or gene mutational data.

11. A medical information processing method, comprising:
receiving omics data, the omics data comprising a plurality of values, wherein each value of the plurality of values is associated with a corresponding biomolecule of a plurality of biomolecules;
calculating a respective distance between each pair of biomolecules from the plurality of biomolecules;
applying a manifold learning method to the distances to obtain a respective position in a two-dimensional space mapped to each biomolecule of the plurality of biomolecules;
adjusting the positions to achieve a more even distribution of the positions over the two-dimensional space; and
storing, in a memory, a two-dimensional image format of display positions for each biomolecule of the plurality of biomolecules based on the adjusted positions.

12. A medical information processing apparatus, the medical information processing apparatus comprising a memory storing a two-dimensional image format of display positions for a plurality of biomolecules, wherein the two-dimensional image format is obtained using the method of claim 11; and
processing circuitry configured to:
receive omics data associated with a subject, the omics data comprising a plurality of values, wherein each value of the plurality of values is associated with a respective biomolecule of the plurality of biomolecules; and
generate a displaying image by mapping each value of the plurality of values onto an image using the two-dimensional image format, based on the display positions in the two-dimensional format.

13. The medical information processing apparatus of claim 12, wherein the processing circuitry is further configured to display the displaying image for human inspection.

14. The medical information processing apparatus of claim 12, wherein the processing circuitry is further configured to input the displaying image to a deep learning algorithm.

15. The medical information processing apparatus of claim 12, wherein the plurality of values in the omics data are normalized.

16. The medical information processing apparatus of claim 15, wherein the displaying image is colored in accordance with the normalized values.

17. The medical information processing apparatus of claim 12, wherein the processing circuitry is further configured to:
provide mouse-over functionality to the displaying image to display the names of the biomolecules mapped to the respective display positions and/or
display functional information related to the biomolecules mapped to the respective display positions.

18. The medical information processing apparatus of claim 12, wherein the processing circuitry is further configured to:
receive further omics data associated with a further subject, each value of the further omics data comprising a further plurality of values, wherein each value of the further plurality of values is associated with a respective biomolecule of the plurality of biomolecules;
generate a further displaying image by mapping each value of the further plurality of image onto a further image using the two-dimensional image format, based on the display positions of the two-dimensional format; and
display the image and further image side by side, or
display the image or further image in a window and allow a user to switch between display of the image and display of the further image.

19. A medical information processing method, the method comprising:
receiving omics data associated with a subject, the omics data comprising a plurality of values, wherein each value of the plurality of values is associated with a respective biomolecule of the plurality of biomolecules; and
generating a displaying image by mapping each value of the plurality of values onto an image using a two-dimensional image format of display positions for a plurality of biomolecules, based on the display position in the two-dimensional image format;
wherein the two-dimensional image format is obtained using the method of claim 11.

20. A medical information processing apparatus comprising:
a memory; and
processing circuitry configured to:
receive omics data, the omics data comprising a plurality of values, wherein each value of the plurality of values is associated with a corresponding biomolecule of a plurality of biomolecules;
calculate a respective distance between each pair of biomolecules from the plurality of biomolecules;
apply a manifold learning method to the distances to obtain a respective position in a two-dimensional space mapped to each biomolecule of the plurality of biomolecules; and
store, in the memory, a two-dimensional image format of display positions for each biomolecule of the plurality of biomolecules based on the positions;
wherein the respective distance between each pair of biomolecules from the plurality of biomolecules is calculated based on information from a biological database.
